(19) United States
(12) Patent Application Publication
(10) Pub. No.: US 2010/0087457 A1
(43) Pub. Date: Apr. 8, 2010

(54) DOSAGES AND METHODS FOR THE TREATMENT OF CANCER

(75) Inventor: MARK LAUGHLIN, Sunnyvale, CA (US)

Correspondence Address: Myriad PHARMACEUTICALS, Inc.;c/o CPA Global, P.O. Box 52050, Minneapolis, MN, 55402, US

(73) Assignee: Myriad Pharmaceuticals, Inc, Salt Lake City, UT (US)

(21) Appl. No.: 12/575,110

(22) Filed: Oct. 7, 2009

Related U.S. Application Data
(63) Continuation of application No. PCT/US2008/059908, filed on Apr. 10, 2008.
(60) Provisional application No. 60/910,956, filed on Apr. 10, 2007.

(51) Int. Cl. A61K 31/517; C07D 241/54

(52) U.S. Cl. 514/266.4; 544/349

(57) ABSTRACT

In general, the invention relates to a pharmaceutical dose having (4-Methoxy-phenyl)-methyl-(2-methyl-quinazolin-4-yl)-amine hydrochloride as the active ingredient that upon administration to a subject provides a Cₘₐₓ of about 1 ng/mL to about 250 ng/mL. The dose may be used in methods of treating cancer through the administration of (4-Methoxy-phenyl)-methyl-(2-methyl-quinazolin-4-yl)-amine hydrochloride. The composition of the invention is formulated with one or more pharmaceutically acceptable excipients, salts or carriers.

### CROSS REFERENCE TO RELATED APPLICATIONS

This application is continuation of International Application No. PCT/US2008/059908, filed Apr. 10, 2008 and published as WO 2008/124824, which claims the benefit of U.S. Provisional Application Ser. No. 60/910,956, filed on Apr. 10, 2007; both of which are incorporated by reference herein in their entirety.

### FIELD OF THE INVENTION

The invention relates to methods and compositions for the treatment of cancer. The invention provides compositions comprising (4-Methoxy-phenyl)-methyl-(2-methyl-quinazolin-4-yl)-amine hydrochloride and one or more liquid diluents and encompasses certain doses and dosing regimens for the treatment of cancer.

### BACKGROUND OF THE INVENTION

Cancer is a common cause of death in the world; about 10 million new cases occur each year, and cancer is responsible for 12% of deaths worldwide, making cancer the third leading cause of death. World Health Organization, National Cancer Control Programmes Policies and Managerial Guidelines (2d ed. 2002).

Despite advances in the field of cancer treatment, the leading therapies to date include surgery, radiation, and chemotherapy. Chemotherapeutic approaches are said to fight cancers that are metastasized or that are particularly aggressive. Most of the cancer chemotherapy agents currently in clinical use are cytotoxins. Cytotoxic agents work by damaging or killing cells that exhibit rapid growth. Ideal cytotoxic agents would have specificity for cancer and tumor cells, while not affecting normal cells. Unfortunately, none have been found and instead agents that target especially rapidly dividing cells (both tumor and normal) have been used.

Accordingly, materials that are cytotoxic to cancer cells while exerting only mild effects on normal cells are highly desirable. In fact, many recent studies have focused on developing alternative anticancer substances capable of specifically suppressing proliferation of tumor cells. Examples of such anticancer compounds may be found in International Pat. Publication No. WO 2005/003100. However, the safety of such compounds and amounts of such compounds that may be safely administered to an individual has not been known. Therefore, there remains a definite need in the art for the discovery of new effective chemotherapeutic agents and dosing ranges that can be administered safely.

### BRIEF SUMMARY OF THE INVENTION

In general, the invention relates to a pharmaceutical composition having (4-Methoxy-phenyl)-methyl-(2-methyl-quinazolin-4-yl)-amine hydrochloride as the active ingredient. More specifically, the invention relates to specific dosage formulations or doses (i.e., unit dosage forms) of (4-Methoxy-phenyl)-methyl-(2-methyl-quinazolin-4-yl)-amine hydrochloride useful in the treatment of cancer, e.g., about 0.3 to about 4.5 mg/m². The compositions of the invention are formulated with one or more pharmaceutically acceptable excipients, salts, or carriers and are delivered intravenously. The (4-Methoxy-phenyl)-methyl-(2-methyl-quinazolin-4-yl)-amine hydrochloride compositions of the invention can be used in methods for treating cancer.

In a first aspect, the invention provides a method of treating an individual with cancer, comprising administering to the individual a therapeutically effective amount of (4-Methoxy-phenyl)-methyl-(2-methyl-quinazolin-4-yl)-amine hydrochloride, or a pharmaceutically acceptable salt, acid or base thereof, sufficient to provide in the individual a plasma Cₘₐₓ (maximum plasma concentration after administration) of about 1 ng/mL to about 250 ng/mL. For example, a dosage comprising (4-Methoxy-phenyl)-methyl-(2-methyl-quinazolin-4-yl)-amine hydrochloride in an amount of no greater than 4.5 mg/m² may be administered. Intravenous administration of a single dose to a subject, provides an estimated Cₘₐₓ of about 14 ng/mL on Day 1 to about 26 ng/mL on Day 15.

A dose of an effective amount, upon administration to a subject, may provide a Cₘₐₓ of about 1 ng/mL to about 150 ng/mL. In a more specific embodiment, said Cₘₐₓ is between about 10 ng/mL and about 30 ng/mL.

In a specific embodiment, the dosage is provided as a pharmaceutical composition composed of (4-Methoxy-phenyl)-methyl-(2-methyl-quinazolin-4-yl)-amine hydrochloride and one or more liquid diluents.

In another aspect, the invention provides a dosage unit of (4-Methoxy-phenyl)-methyl-(2-methyl-quinazolin-4-yl)-amine hydrochloride, or a pharmaceutically acceptable salt, acid or base thereof, in a quantity of less than about 30 mg, such as about 1 mg to about 20 mg, or about 5 mg to about 20 mg. Such dosage unit may be provided in a kit or vial.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

In general, the invention relates to a pharmaceutical composition having (4-Methoxy-phenyl)-methyl-(2-methyl-quinazolin-4-yl)-amine hydrochloride as the active ingredient. The invention encompasses intravenous compositions that, upon administration of a dose of said pharmaceutical composition to a subject, provides pharmacokinetic and therapeutic characteristics particularly useful in the methods of the invention. The invention also encompasses the use of the inventive composition according to the treatment regimens of the invention by an individual desiring or needing such treatment, thus providing a treatment of cancer. The composition of the invention is formulated with one or more pharmaceutically acceptable excipients, salts, or carriers. The (4-Methoxy-phenyl)-methyl-(2-methyl-quinazolin-4-yl)-amine hydrochloride composition of the invention can be used in methods for treating cancer, which is a group of diseases characterized by the uncontrolled growth and spread of abnormal cells.

Such diseases include, but are not limited to, Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

As used herein, the term “dosage unit” refers to a physically discrete unit, suitable as a unitary dosage for a human patient. Each unit contains a predetermined range of (4-Methoxy-phenyl)-methyl-(2-methyl-quinazolin-4-yl)-amine hydrochloride that was discovered as a result of this invention to be useful for administration in achieving the desired pharmacokinetic profile which yields the desired therapeutic effect. The dosage unit is composed of (4-Methoxy-phenyl)-methyl-(2-methyl-quinazolin-4-yl)-amine hydrochloride optionally associated with one or more liquid diluents.

As used herein, the term “dose” or “dosage” refers the amount of active ingredient that an individual takes or is administered at one time or over a specified period of time. For example, a 3.3 mg/m² dose of (4-Methoxy-phenyl)-methyl-(2-methyl-quinazolin-4-yl)-amine hydrochloride may be administered weekly through an infusion process that lasts 0.5, 1, 2, 3, 4, or 8 hours.

### Dosages

The invention is based on the discovery that a dosage having (4-Methoxy-phenyl)-methyl-(2-methyl-quinazolin-4-yl)-amine hydrochloride in an amount of no greater than about 4.5 mg/m² provides a PK profile believed to be effective in treating cancer. Without wishing to be bound by theory, it is believed that the PK profile obtained maximizes therapeutic effects while minimizing side-effects thereby providing maximum benefit to the patient. The dose can be administered over 0.5, 1, 2, 3, 4, 8, or more hours where the dose is about no greater than about 4.5 mg/m². In certain embodiments, (4-Methoxy-phenyl)-methyl-(2-methyl-quinazolin-4-yl)-amine hydrochloride is administered at a dose of not more than about 3.3 mg/m². In some embodiments, (4-Methoxy-phenyl)-methyl-(2-methyl-quinazolin-4-yl)-amine hydrochloride is administered at a dose of not more than about 2.7 mg/m². In further embodiments, (4-Methoxy-phenyl)-methyl-(2-methyl-quinazolin-4-yl)-amine hydrochloride is administered at a dose of not more than about 2.1 mg/m². For example, the dose may be from about 0.3 to about 3.3 mg/m². For example, the dose may be from about 2.7 to about 3.3 mg/m².

In one embodiment, administration of a dose to a subject, provides a Cₘₐₓ of about 1 ng/mL to about 150 ng/mL per dose, and, preferably, between 10 ng/mL to about 30 ng/mL per mL per dose. Administration of a single dose of the compositions of the invention to a subject provides an AUC (area under curve of concentration versus time; total drug exposure) of from about 20 hr·ng/mL to about 650 hr·ng/mL, and preferably from about 20 hr·ng/mL to about 150 hr·ng/mL.

In another embodiment, the invention provides a dosage unit of (4-Methoxy-phenyl)-methyl-(2-methyl-quinazolin-4-yl)-amine hydrochloride, or a pharmaceutically acceptable salt, acid or base thereof, in a quantity of less than about 30 mg, such as about 1 mg to about 20 mg, or about 5 mg to about 20 mg. In a specific embodiment, a dosage unit of (4-Methoxy-phenyl)-methyl-(2-methyl-quinazolin-4-yl)-amine hydrochloride, is provided in a quantity of about 5 mg, about 10 mg, or about 20 mg. Such dosage units may be provided in a kit or vial.

Such dosage unit is dependent upon the amount of drug administered to the patient, and factors such as the size and weight of the patient. For example, if a specific dose is desired according to the volume of the patient, such as about 3.3 mg/m², the height and weight of a patient may be taken and converted to a volume measurement. Although the range of volumes for human patients varies, the dose ranges found to be safely tolerated of less than about 3.3 mg/m² reveal that a quantity of less than about 30 mg, such as about 1 mg to about 20 mg, or about 5 mg to about 20 mg is a sufficient dosage unit for most patients being administered (4-Methoxy-phenyl)-methyl-(2-methyl-quinazolin-4-yl)-amine hydrochloride. Additional dosage units may include between about 0.5 mg to about 15 mg, such as about 2 mg to about 10 mg, or about 4 mg to about 8 mg. For example, a dosage unit of 4-Methoxy-phenyl)-methyl-(2-methyl-quinazolin-4-yl)-amine hydrochloride may be about 2, about 3, about 4, about 5, about 6, about 7, or about 8 mg.

### PK_Profile

The present invention provides for the administration of (4-Methoxy-phenyl)-methyl-(2-methyl-quinazolin-4-yl)-amine hydrochloride to an individual having cancer, so as to obtain a desired pharmacokinetic profile, for example, a desired concentration of (4-Methoxy-phenyl)-methyl-(2-methyl-quinazolin-4-yl)-amine hydrochloride in the plasma over a period of time. Such preferred pharmacokinetic profiles and/or endpoints may be achieved through the administration of specific doses, for example, a dose of no greater than 4.5 mg/m² such as a range of about 2.1 to about 3.3 mg/m², or may be achieved through the administration of doses individually-tailored for the specific recipient, taking into account factors such as weight, percent body fat, metabolism, etc.

Thus, in one embodiment, the invention provides for a method of administering (4-Methoxy-phenyl)-methyl-(2-methyl-quinazolin-4-yl)-amine hydrochloride to an individual, wherein said (4-Methoxy-phenyl)-methyl-(2-methyl-quinazolin-4-yl)-amine hydrochloride is administered in an amount sufficient to result in a plasma Cₘₐₓ of about 1 ng/mL to about 250 ng/mL, and wherein said individual has cancer. In a more specific embodiment, said plasma Cₘₐₓ is from about 10 ng/mL to about 30 ng/mL. In another more specific embodiment, said Cₘₐₓ is from about 30 ng/mL to about 150 ng/mL. In another embodiment, said Cₘₐₓ is between about 1 ng/mL and about 10 ng/mL. In a more specific embodiment, said plasma Cₘₐₓ is from about 5 ng/mL to about 100 ng/mL.

(4-Methoxy-phenyl)-methyl-(2-methyl-quinazolin-4-yl)-amine hydrochloride levels in the plasma or in the cerebrospinal fluid may be assessed by any art-accepted method. Determination of the concentration of (4-Methoxy-phenyl)-methyl-(2-methyl-quinazolin-4-yl)-amine hydrochloride in cerebrospinal fluid may be accomplished as follows. Cerebrospinal fluid containing (4-Methoxy-phenyl)-methyl-(2-methyl-quinazolin-4-yl)-amine hydrochloride and an internal standard, for example, (4-Methoxy-phenyl)-methyl-(2-methyl-quinazolin-4-yl)-amine hydrochloride-D₃, is mixed with mobile phase and centrifuged. The supernatant is then transferred to a 96-well block and an aliquot of extract is injected onto a Micromass Ultima LC-MS-MS equipped with an enantio-selective column. Peak area of the (4-Methoxy-phenyl)-methyl-(2-methyl-quinazolin-4-yl)-amine hydrochloride product ion is measured against the peak area of the (4-Methoxy-phenyl)-methyl-(2-methyl-quinazolin-4-yl)-amine hydrochloride-D₃ internal standard product ion. Quantification may be performed using a weighted (1/x²) linear least squares regression analysis for each enantiomer generated from fortified plasma standards prepared in bulk and frozen.

### Treatment Method

In an embodiment, the invention provides a method of treating cancer, which is a group of diseases characterized by the uncontrolled growth and spread of abnormal cells. Such diseases include, but are not limited to, Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

In a specific embodiment, the method of treating cancer comprises administering to a patient in need of such treatment, a dose of a pharmaceutical composition comprising an effective amount of (4-Methoxy-phenyl)-methyl-(2-methyl-quinazolin-4-yl)-amine hydrochloride, wherein a dose of an effective amount upon administration to a subject provides a Cₘₐₓ of about 1 ng/mL to about 250 ng/mL per dose, or about 10 ng/mL to about 150 ng/mL per dose. The dose can be administered over 0.5, 1, 2, 3, 4, 8, or more hours where the dose is no greater than about 4.5 mg/m², such as not more than 3.3 mg/m² or not more than about 2.1 mg/m². The dose may be from about 0.3 to about 3.3 mg/m². For example, the dose may be from about 2.7 to about 3.3 mg/m².

For example, (4-Methoxy-phenyl)-methyl-(2-methyl-quinazolin-4-yl)-amine hydrochloride may be administered for treatment of cancer at an amount of between about 0.5 mg to about 15 mg, such as about 2 mg to about 10 mg, or about 4 mg to about 8 mg. In certain embodiments, 4-Methoxy-phenyl)-methyl-(2-methyl-quinazolin-4-yl)-amine hydrochloride is administered at an amount of not more than about 10 mg, such as not more than about 8 mg or not more than about 6 mg. In additional embodiments, 4-Methoxy-phenyl)-methyl-(2-methyl-quinazolin-4-yl)-amine hydrochloride is administered at an amount of about 2, about 3, about 4, about 5, about 6, about 7, or about 8 mg.

The active compound(s) may be administered to a subject over various time frames and for varying lengths. For example, active compounds that are infused may be administered through an infusion process that last 0.5, 1, 2, 3, 4, or 8 hours. Additionally, the active compounds may be administered daily, weekly, monthly, or according to various schedules such as cycles of once a week for three weeks followed by a week of no administration. For example, (4-Methoxy-phenyl)-methyl-(2-methyl-quinazolin-4-yl)-amine hydrochloride may be administered once every two weeks on a six week cycle. Alternatively, (4-Methoxy-phenyl)-methyl-(2-methyl-quinazolin-4-yl)-amine hydrochloride may be administered on an eight week schedule with (4-Methoxy-phenyl)-methyl-(2-methyl-quinazolin-4-yl)-amine hydrochloride once every week for six weeks followed by no administration for two weeks.

The pharmacokinetic parameters referred to herein are based on the averages for a group of about 3 or more individuals for each dosing regimen. The skilled artisan understands that individuals will vary and can have pharmacokinetic parameters outside the given ranges. Similarly, the efficacy or therapeutic endpoint parameters are based on averages for a group of individuals and individuals experience efficacies that fall outside the given ranges.

### Formulations

The present invention also includes methods comprising administering to an animal an effective amount of (4-Methoxy-phenyl)-methyl-(2-methyl-quinazolin-4-yl)-amine hydrochloride, or a pharmaceutically acceptable salt or prodrug thereof, and one or more liquid diluents. Such compositions include compositions disclosed in PCT Pub. No. WO 2006/138608, and may be manufactured according to the methods disclosed therein, the relevant portions of which are incorporated herein by reference.

Also included within the scope of the present invention are the non-toxic pharmaceutically acceptable salts of the compounds of the present invention. Acid addition salts are formed by mixing a solution of the compounds of the present invention with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Basic salts are formed by mixing a solution of the compounds of the present invention with a solution of a pharmaceutically acceptable non-toxic base, such as sodium hydroxide, potassium hydroxide, choline hydroxide, sodium carbonate, Tris, N-methyl-glucamine and the like.

The pharmaceutical compositions of the invention may be administered to any animal, which may experience the beneficial effects of the compounds of the invention. Foremost among such animals are mammals, e.g., humans and veterinary animals, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

### PREPARATION of (4-Methoxy-phenyl)-methyl-(2-methyl-quinazolin-4-yl)-amine hydrochloride

In practicing the present invention, (4-Methoxy-phenyl)-methyl-(2-methyl-quinazolin-4-yl)-amine hydrochloride may be prepared using methods known to those skilled in the art. Specifically, (4-Methoxy-phenyl)-methyl-(2-methyl-quinazolin-4-yl)-amine hydrochloride may be prepared according to International Pat. Publication No. WO 2005/003100 and as illustrated by the exemplary reaction in Scheme 1 below.

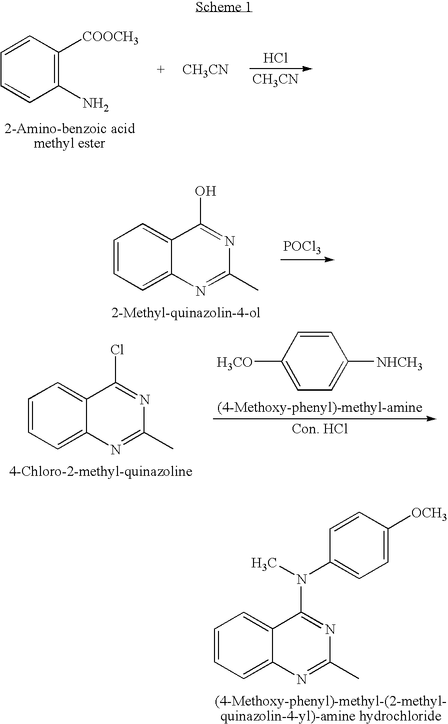

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### Example 1

### Preparation of (4-Methoxy-phenyl)-methyl-(2-methyl-quinazolin-4-yl)-amine hydrochloride

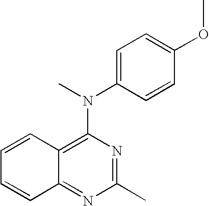

### (4-Methoxy-phenyl)-methyl-(2-methyl-quinazolin-4-yl)-amine hydrochloride

a) 4-Chloro-2-methyl-quinazoline: A stirred suspension of 2-methyl-4(3H)-quinazolinone (5 g, 31.2 mmol) in POCl₃ (100 mL) was heated at 120° C. for 3 h. The excess POCl₃ was removed under vacuum, then to the residue was added crushed ice and 200 mL of saturated NaHCO₃, and the mixture was extracted with ethyl acetate (200 mL×2). The combined extracts were washed with water, saturated NaCl, dried over anhydrous MgSO₄, filtered and concentrated. The crude product was purified by column chromatography (5-8% ethyl acetate/hexane) to give the title compound (2.5 g, 14.0 mmol, 45%). ¹H NMR (CDCl₃): 8.21-8.25 (m, 1H), 7.89-7.99 (m, 2H), 7.66 (ddd, 1H, J=1.8, 6.6, 8.7), 2.87 (s, 3H).

b) (4-Methoxy-phenyl)-methyl-(2-methyl-quinazolin-4-yl)-amine hydrochloride: The title compound was prepared from 4-chloro-2-methyl-quinazoline (2.31 g, 12.9 mmol) and (4-methoxy phenyl)-methyl-amine (2.0 g, 14.6 mmol) by a procedure similar to example 1b and was isolated as solids (2.90 g, 9.18 mmol, 71%). ¹H NMR (CDCl₃): 8.53 (dd, 1H, J=0.6, 8.1), 7.7 (ddd, 1H, J=1.2, 7.2, 8.4), 7.22 (m, 2H), 7.13 (ddd, 1H, J=1.2, 7.2, 8.7), 7.05 (m, 2H), 6.76 (d, 1H, J=8.7), 3.91 (s, 3H), 3.78 (s, 3H), 2.96 (s, 3H).

### Example 2

### Pharmaceutical Composition

A pharmaceutical composition is prepared by combining and mixing 100 grams of (4-Methoxy-phenyl)-methyl-(2-methyl-quinazolin-4-yl)-amine hydrochloride and 1 gram of BHT and dissolving into 10 liters of D5W with the pH adjusted to pH=5 with hydrochloric acid. This solution is sterile filtered using a 0.2 μm Teflon filter (PTFE).

### Example 3

### Pharmaceutical Composition

A pharmaceutical composition was formed by dissolving 300.1 grams (4-Methoxy-phenyl)-methyl-(2-methyl-quinazolin-4-yl)-amine hydrochloride into 13.652 kg surfactant (CREMOPHOR® EL) and 13.652 kg viscosity reducing agent (ethanol 190 proof). This solution was sterile filtered through a 0.2 μm Millipore Durapore filter (PVDF), and packaged into 10 ml sterile glass vials.

### Example 4

### Pharmaceutical Composition

A pharmaceutical composition was formed by dissolving 300.1 grams (4-Methoxy-phenyl)-methyl-(2-methyl-quinazolin-4-yl)-amine hydrochloride and 30.12 grams antioxidant (BHT) into 13.652 kg surfactant (CREMOPHOR® EL) and 13.652 kg viscosity reducing agent (ethanol 190 proof). This solution was sterile filtered through a 0.2 μm Millipore Durapore filter (PVDF), and packaged into 10 ml sterile glass vials.

### Example 5

### Pharmaceutical Composition

A pharmaceutical composition is formed by dissolving 300.1 grams (4-Methoxy-phenyl)-methyl-(2-methyl-quinazolin-4-yl)-amine hydrochloride and 30.12 grams antioxidant (BHT) into 13.652 kg surfactant (CREMOPHOR® EL) and 11.652 kg viscosity reducing agent (ethanol 190 proof), and 2 kg WFI (water for injection). This solution is sterile filtered through a 0.2 μm Millipore Durapore filter (PVDF), and packaged into 10 ml sterile glass vials.

### Example 6

### Method of Administration

About 0.01 ml to about 50 ml of the pharmaceutical composition of Example 5 is accurately measured and then added to an i.v. bag containing about 100 ml to about 1000 ml of sterile dextrose 5% in water (D5W). The amount of pharmaceutical composition and D5W used varies according to the desired therapeutic dose and size of the patient. The resulting mixture is then parenterally infused into the patient.

### Example 7

### Phase I Clinical Trial of Administration of (4-Methoxy-phenyl)-methyl-(2-methyl-quinazolin-4-yl)-amine hydrochloride for Subjects with Refractory Solid Tumors

An open-label, dose-escalating, multiple-dose study to define the safety, tolerability and pharmacokinetics of weekly intravenous administration of (4-Methoxy-phenyl)-methyl-(2-methyl-quinazolin-4-yl)-amine hydrochloride was performed. A dosing schedule (each 4 week cycle) was performed for (4-Methoxy-phenyl)-methyl-(2-methyl-quinazolin-4-yl)-amine hydrochloride weekly for 3 weeks with no infusion on the fourth week of each cycle. Subjects with refractory solid tumors were enrolled in cohorts of 3. During Cycle 1, subjects were hospitalized during each infusion of (4-Methoxy-phenyl)-methyl-(2-methyl-quinazolin-4-yl)-amine hydrochloride and remained for observation and safety evaluation for approximately 24 hours following the end of the infusion. All subjects had continuous telemetry for 2 hours prior to infusion, for 1-2 hour infusion and for 3 hours after the end of the infusion. Any clinically significant electrocardiographic (ECG) wave form abnormality was recorded and prolongation of the monitoring period extended at the discretion of the principal investigator.

Electrocardiograms were obtained prior to starting the infusion and within 30 minutes of the end of infusion for each infusion of the first cycle. Electrocardiograms on Day 1 were obtained in triplicate 5 minutes apart.

Neurocognitive assessments were made by administration of the Mini-Mental State Examination (MMSE), the Hopkins Verbal Learning and timed Grooved Pegboard tests before administration of the intravenous infusion and approximately 24 hours of the infusion at each weekly administration of the first cycle.

On days 1, 8, and 15 of each cycle, vital signs were obtained prior to the first dose, at 15, 30, and 60 minutes after the initiation of the infusion, and at 0.5, 1, 1.5, 2, and 4 hours after the end of the intravenous infusion. Vital signs at all time points beyond the start of the intravenous infusion included heart rate, blood pressure and respirations. Temperature was measured at the end of the infusion and 4 hours later.

Individual subjects were allowed to continue on repeated weekly×3 administrations every 28 days with no dose increase provided there was no unacceptable toxicity or disease progression.

Tumor response was evaluated by response evaluation criteria in solid tumors (RECIST) criteria. To prevent sever hypersensitivity reactions due to Cremophor® EL, subjects were premedicated with oral dexamethasone (20 mg) administered approximately 12 and 6 hours before the intravenous infusion with (4-Methoxy-phenyl)-methyl-(2-methyl-quinazolin-4-yl)-amine hydrochloride, diphenhydramine (50 mg) or its equivalent administered intravenously 30-60 minutes before (4-Methoxy-phenyl)-methyl-(2-methyl-quinazolin-4-yl)-amine hydrochloride, and cimetidine (300 mg) or ranitidine (50 mg) administered intravenously 30-60 minutes before (4-Methoxy-phenyl)-methyl-(2-methyl-quinazolin-4-yl)-amine hydrochloride.

Dose escalation of subjects proceeded sequentially as presented in Table 1 below:

TABLE 1

| Cohort Number | Dose level (modified Fibonacci series) |
|---|---|
| Cohort 1 | Dose 1 = 0.3 mg/m² |
| Cohort 2 | Dose 2 = 0.6 mg/m² |
| Cohort 3 | Dose 3 = 1.0 mg/m² |
| Cohort 4 | Dose 4 = 1.5 mg/m² |
| Cohort 5 | Dose 5 = 2.1 mg/m² |
| Cohort 6 | Dose 6 = 2.7 mg/m² |
| Cohort 7 | Dose 7 = 3.3 mg/m² |

Blood samples were collected prior to dosing, at the end of the infusion, and 1, 2, 4, 6, 12, and 24 hours after the infusion. This sampling schedule occurred on Days 1 and 15 with administration of the first and third doses. Samples were collected into tubes containing EDTA and stored on ice until centrifuged. The plasma was frozen at approximately −20° C. in labeled, plastic tubes, or vials, and tightly capped. The samples were shipped to Myriad Pharmaceuticals, Inc. for analysis of (4-Methoxy-phenyl)-methyl-(2-methyl-quinazolin-4-yl)-amine hydrochloride. Pharmacokinetic analysis was conducted using WinNonlin 4.0®. The pharmacokinetic data are presented in Tables 2 and 3 below:

TABLE 2
Plasma Concentrations (ng/mL) of (4-Methoxy-phenyl)-methyl-
(2-methyl-quinazolin-4-yl)-amine hydrochloride on Day 1

| | Infusion | | | | | | | | | |
|---|---|---|---|---|---|---|---|---|---|---|
| Dose | Length | | Collection Time | | | | | | | |
| (mg/m²) | (hr) | Subject | PREDOSE | EOI | 1 HR | 2 HR | 4 HR | 6 HR | 12 HR | 24 HR |
| 0.3 | 1 | 0101-1 | BQL | 1.52 | 1.17 | BQL | BQL | BQL | BQL | BQL |
| | | 0102-4 | BQL | 2.43 | 1.25 | BQL | BQL | BQL | BQL | BQL |
| | | 0103-0 | BQL | 1.77 | 1.03 | BQL | BQL | BQL | BQL | BQL |
| 0.6 | 1 | 0201-6 | BQL | 20.05 | 8.78 | 4.59 | 4.04 | 2.59 | 1.30 | BQL |
| | | 0202-8 | BQL | 3.07 | 1.47 | BQL | BQL | BQL | BQL | BQL |
| | | 0203-7 | BQL | 74.15 | 40.85 | 26.73 | 12.58 | 7.90 | 8.23 | 5.89 |
| 1.0 | 1 | 0301-4 | BQL | 11.36 | 1.64 | BQL | 1.04 | BQL | BQL | BQL |
| | | 0302-2 | BQL | 86.49 | 15.31 | 16.85 | 3.76 | 3.67 | 3.62 | 1.98 |
| | | 0304-6 | BQL | 3.29 | 3.61 | 2.16 | 1.60 | 1.17 | BQL | BQL |
| 1.5 | 1 | 0401-2 | BQL | 6.50 | 3.18 | 2.84 | 1.69 | 1.03 | BQL | BQL |
| | | 0402-7 | BQL | 9.59 | 2.69 | 1.77 | BQL | 1.03 | BQL | BQL |
| | | 0403-6 | BQL | 114.91 | 7.69 | 9.27 | 3.69 | 4.39 | 1.95 | BQL |
| | | 0404-3 | BQL | 4.69 | 2.70 | 2.28 | 1.47 | BQL | BQL | BQL |
| | | 0405-8 | BQL | 4.22 | 1.95 | 1.30 | BQL | BQL | BQL | BQL |
| | | 0406-2 | BQL | 7.28 | 2.53 | 1.75 | BQL | BQL | BQL | BQL |
| | | 0407-0 | BQL | 4.98 | 2.33 | 2.22 | 1.25 | BQL | BQL | BQL |
| 2.1 | 1 | 0503-2 | BQL | 19.04 | 4.06 | 3.28 | 2.64 | 1.12 | BQL | BQL |
| | | 0505-7 | BQL | 14.34 | 5.53 | 4.78 | 2.69 | 1.62 | BQL | BQL |
| | | 0506-1 | BQL | 6.32 | 3.75 | 3.94 | 2.41 | 1.18 | 1.09 | BQL |
| 2.7 | 1 | 0602-5 | BQL | 10.42 | 6.16 | 4.37 | 3.16 | 2.09 | BQL | BQL |
| | | 0603-9 | BQL | 11.10 | 4.69 | 4.03 | 2.33 | 1.68 | BQL | BQL |
| | | 0604-6 | BQL | 7.52 | NSR | 5.54 | 3.91 | 3.11 | 2.35 | 1.36 |
| 3.3 | 1 | 0701-0 | BQL | 8.94 | 6.45 | 4.82 | 2.49 | 1.57 | BQL | BQL |
| | | 0702-3 | BQL | 10.67 | 7.25 | 7.71 | 4.84 | 3.12 | 1.22 | BQL |
| | | 0703-4 | BQL | 8.53 | 5.80 | 5.87 | 3.29 | 2.50 | 2.23 | BQL |
| | | 0704-7 | BQL | 14.84 | 10.52 | 8.50 | 6.76 | 4.99 | 2.09 | 1.16 |
| | | 0705-2 | BQL | 31.16 | 14.93 | 11.50 | 8.78 | 5.73 | 2.91 | 3.20 |
| 3.3 | 2 | 0907-8 | BQL | 8.67 | 4.59 | 6.03 | 4.73 | 3.35 | 1.07 | BQL |
| | | 1001-5 | BQL | 11.97 | 10.36 | 10.37 | 7.72 | 5.37 | 4.13 | 1.73 |
| | | 1002-7 | BQL | 13.37 | 9.72 | 8.81 | 5.96 | 4.96 | 2.32 | BQL |
| | | 1003-6 | BQL | 16.77 | 6.63 | 5.19 | 2.65 | 2.32 | BQL | BQL |
| 3.9 | 2 | 0801-7 | BQL | 7.52 | 5.99 | 6.71 | 3.56 | 4.23 | 2.83 | 1.12 |
| | | 0802-9 | BQL | 19.18 | 12.05 | 9.85 | 3.67 | 3.34 | 2.16 | BQL |
| | | 0803-4 | BQL | 23.81 | 9.11 | 11.84 | 20.00 | 11.39 | 7.29 | 3.76 |
| | | 0805-5 | BQL | 13.84 | NSR | NSR | 5.36 | 2.91 | 1.33 | BQL |
| | | 0806-4 | BQL | 13.39 | 8.26 | 5.58 | 3.77 | 2.64 | 3.02 | BQL |
| | | 0807-7 | BQL | 22.47 | 11.89 | 8.73 | 7.13 | 5.23 | 2.19 | BQL |
| 4.5 | 2 | 0901-3 | BQL | 93.79 | 12.84 | 6.34 | 8.17 | 6.51 | 3.80 | 1.17 |
| | | 0902-6 | BQL | 17.96 | 7.90 | 6.25 | 3.32 | 2.25 | BQL | BQL |
| | | 0903-2 | BQL | 181.07 | 16.98 | 13.24 | 14.43 | 10.48 | 4.58 | 1.11 |
| | | 0905-0 | BQL | 41.63 | 13.06 | 10.78 | 6.12 | 5.44 | 1.66 | BQL |
| | | 0906-6 | BQL | 14.36 | 11.02 | 10.24 | 9.72 | 7.01 | 1.71 | 1.07 |

BQL = Below Quantitation Limits
NSR = No Sample Received
Note:
Day 1 Predose BQL values were set to zero for analysis, all other BQL values were set to the LLOQ (1 ng/mL)

TABLE 3
Plasma Concentrations (ng/mL) of (4-Methoxy-phenyl)-methyl-
(2-methyl-quinazolin-4-yl)-amine hydrochloride on Day 15

| | Infusion | | | | | | | | | |
|---|---|---|---|---|---|---|---|---|---|---|
| Dose | Length | | Collection Time | | | | | | | |
| (mg/m²) | (hr) | Subject | PREDOSE | EOI | 1 HR | 2 HR | 4 HR | 6 HR | 12 HR | 24 HR |
| 0.3 | 1 | 0101-1 | BQL | 4.11 | 1.43 | BQL | BQL | BQL | BQL | BQL |
| | | 0102-4 | BQL | 1.56 | BQL | BQL | BQL | BQL | BQL | BQL |
| | | 0103-0 | BQL | 2.54 | BQL | BQL | BQL | BQL | BQL | BQL |
| 0.6 | 1 | 0201-6 | BQL | 38.28 | 7.10 | 4.47 | 4.59 | 3.93 | 2.41 | 1.12 |
| | | 0203-7 | BQL | 253.50 | 54.92 | 34.68 | 54.09 | 32.48 | 73.17 | 8.08 |
| 1.0 | 1 | 0301-4 | BQL | 8.27 | 2.24 | 1.23 | BQL | BQL | BQL | BQL |
| | | 0304-6 | BQL | 7.82 | 2.51 | 2.15 | 1.26 | BQL | BQL | NSR |
| 1.5 | 1 | 0402-7 | BQL | 4.22 | 2.54 | 1.78 | 1.01 | BQL | BQL | BQL |
| | | 0403-6 | BQL | NSR | NSR | NSR | NSR | NSR | NSR | NSR |
| | | 0404-3 | BQL | 8.49 | 4.40 | 2.74 | 1.60 | 1.47 | 2.24 | 1.63 |
| | | 0405-8 | BQL | 6.68 | 7.43 | 4.43 | 1.40 | 1.71 | BQL | BQL |
| | | 0406-2 | BQL | 16.35 | 3.29 | 3.46 | 1.30 | BQL | BQL | BQL |
| 2.1 | 1 | 0503-2 | BQL | 16.65 | 4.81 | 3.04 | 2.05 | 1.25 | BQL | BQL |
| | | 0505-7 | BQL | 38.28 | 6.21 | 6.27 | NSR | 3.95 | 1.09 | 2.63 |
| | | 0506-1 | BQL | 12.64 | 7.19 | 5.84 | 3.79 | 2.88 | 1.52 | BQL |
| 2.7 | 1 | 0602-5 | BQL | 10.19 | 5.32 | 3.94 | 2.73 | 1.67 | BQL | BQL |
| | | 0603-9 | BQL | 106.72 | 9.36 | 14.98 | 2.68 | 1.80 | BQL | 1.00 |
| | | 0604-6 | BQL | 139.19 | 27.95 | 24.04 | 6.96 | 12.19 | 3.95 | 4.75 |
| 3.3 | 1 | 0701-0 | 5.79 | 29.19 | 14.65 | 12.79 | 9.79 | 7.53 | 6.94 | 5.02 |
| | | 0702-3 | BQL | 10.36 | 9.00 | 6.82 | 4.53 | 3.91 | 2.27 | 1.39 |
| | | 0704-7 | BQL | 22.68 | 17.56 | 15.34 | 7.37 | 12.09 | 3.54 | NSR |
| | | 0705-2 | BQL | 50.25 | 15.17 | 12.35 | 8.57 | 12.67 | 5.86 | NSR |
| 3.3 | 2 | 0907-8 | BQL | 11.96 | 6.84 | 3.80 | 4.21 | 3.23 | BQL | BQL |
| | | 1001-5 | BQL | 21.49 | 12.21 | 7.50 | 9.69 | 8.43 | 2.58 | 1.48 |
| | | 1002-7 | BQL | 9.21 | 6.40 | 5.52 | 4.50 | 2.95 | 1.54 | BQL |
| | | 1003-6 | BQL | 10.62 | 7.21 | 4.16 | 2.93 | 1.67 | 1.00 | NSR |
| 3.9 | 2 | 0802-9 | BQL | 117.38 | 10.56 | 10.42 | 8.24 | 6.62 | 1.87 | 1.80 |
| | | 0803-4 | BQL | 14.83 | 10.97 | 8.48 | 9.13 | 2.82 | 60.12 | 22.12 |
| | | 0805-5 | 1.51 | 22.63 | 12.15 | 14.58 | 7.84 | 5.80 | 5.12 | 3.26 |
| | | 0806-4 | BQL | 9.33 | 7.79 | 7.36 | 10.17 | 5.55 | 3.16 | 1.67 |
| | | 0807-7 | 1.24 | 27.10 | 9.85 | 8.35 | 6.81 | NSR | NSR | NSR |
| 4.5 | 2 | 0901-3 | BQL | 21.52 | 13.60 | 13.07 | 10.26 | 8.06 | 3.86 | 1.57 |
| | | 0902-6 | 4.43 | 111.60 | 17.94 | 14.46 | 7.80 | 8.43 | 2.81 | 3.54 |
| | | 0903-2 | BQL | 23.49 | 11.45 | 12.32 | 6.81 | 4.50 | 2.59 | 1.03 |
| | | 0905-0 | BQL | 52.66 | 10.03 | 8.11 | 5.01 | 3.50 | 1.63 | BQL |

BQL = Below Quantitation Limits
NSR = No Sample Received
Note:
Day 15 BQL values were set to the LLOQ (1 ng/mL) for analysis

The results of the Phase 1 Trial show that there is no evidence of cytotoxity peripherally at the administered doses. There were incidences of intratumor bleeding and the dose limiting toxicity was demonstrated to be vascular in nature, manifested by an acute coronary syndrome. There were no significant effects on cardiac conduction (PR, QRS or QTc) but there was a dose-related increase in systolic blood pressure and occasional episodes of bradycardia. Accordingly, (4-Methoxy-phenyl)-methyl-(2-methyl-quinazolin-4-yl)-amine hydrochloride is thus shown to be safe and tolerable.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. A method of treating an individual with cancer, comprising administering to said individual a therapeutically effective amount of (4-Methoxy-phenyl)-methyl-(2-methyl-quinazolin-4-yl)-amine hydrochloride, sufficient to provide in the individual a plasma Cₘₐₓ of about 1 ng/mL to about 250 ng/mL, or an equimolar amount of (4-Methoxy-phenyl)-methyl-(2-methyl-quinazolin-4-yl)-amine, or an equimolar amount of a pharmaceutically acceptable salt, acid, or base of (4-Methoxy-phenyl)-methyl-(2-methyl-quinazolin-4-yl)-amine.

2. The method of claim 1, wherein said therapeutically effective amount of (4-Methoxy-phenyl)-methyl-(2-methyl-quinazolin-4-yl)-amine hydrochloride is no greater than about 4.5 mg/m².

3. The method of claim 1, wherein said therapeutically effective amount of (4-Methoxy-phenyl)-methyl-(2-methyl-quinazolin-4-yl)-amine hydrochloride is no greater than about 3.3 mg/m².

4. The method of claim 1, wherein said therapeutically effective amount of (4-Methoxy-phenyl)-methyl-(2-methyl-quinazolin-4-yl)-amine hydrochloride is no greater than about 2.7 mg/m².

5. The method of claim 1, wherein said therapeutically effective amount of (4-Methoxy-phenyl)-methyl-(2-methyl-quinazolin-4-yl)-amine hydrochloride is no greater than about 2.1 mg/m².

6. The method of claim 1, wherein said therapeutically effective amount of (4-Methoxy-phenyl)-methyl-(2-methyl-quinazolin-4-yl)-amine hydrochloride is from about 0.3 to about 4.5 mg/m².

7. The method of claim 1, wherein said therapeutically effective amount of (4-Methoxy-phenyl)-methyl-(2-methyl-quinazolin-4-yl)-amine hydrochloride is from about 0.3 to about 3.3 mg/m².

8. The method of claim 1, wherein said therapeutically effective amount of (4-Methoxy-phenyl)-methyl-(2-methyl-quinazolin-4-yl)-amine hydrochloride is from about 2.1 to about 3.3 mg/m².

9. The method of claim 1, wherein said Cₘₐₓ is from about 10 ng/mL to about 150 ng/mL.

10. The method of claim 1, wherein said Cₘₐₓ is from about 10 ng/mL to about 30 ng/mL.

11. The method of claim 1, said Cₘₐₓ if from about 1 ng/mL and about 10 ng/mL.

12. The method of claim 1, said Cₘₐₓ if from Cₘₐₓ is from about 5 ng/mL to about 100 ng/mL.

13. The method of claim 1, further comprising analyzing said individuals cerebrospinal fluid to determine a level of (4-Methoxy-phenyl)-methyl-(2-methyl-quinazolin-4-yl)-amine hydrochloride, the free acid thereof, a metabolite thereof, or a pharmaceutically acceptable salt, acid, or base of the free acid thereof.

14. A dosage unit of (4-Methoxy-phenyl)-methyl-(2-methyl-quinazolin-4-yl)-amine hydrochloride in a quantity of less than about 30 mg.

15. The dosage unit of claim 14 wherein said quantity of (4-Methoxy-phenyl)-methyl-(2-methyl-quinazolin-4-yl)-amine hydrochloride is from about 1 mg to about 20 mg.

16. The dosage unit of claim 14 wherein said quantity of (4-Methoxy-phenyl)-methyl-(2-methyl-quinazolin-4-yl)-amine hydrochloride is from about 5 mg to about 20 mg.

17. The dosage unit of claim 14, wherein said quantity of 4-Methoxy-phenyl)-methyl-(2-methyl-quinazolin-4-yl)-amine hydrochloride is about 2, about 3, about 4, about 5, about 6, about 7, or about 8 mg.

18. The dosage unit of claim 14 wherein such dosage unit is provided in a kit or vial.

19. A method of treating an individual with cancer, comprising administering to said individual a therapeutically effective amount of (4-Methoxy-phenyl)-methyl-(2-methyl-quinazolin-4-yl)-amine hydrochloride, or an equimolar amount of the free acid thereof or an equimolar amount of pharmaceutically acceptable salt, acid, or base of the free acid thereof, sufficient to provide in the individual a total drug exposure of from about 20 hr·ng/mL to about 650 hr·ng/mL.

20. The method of claim 19, wherein said total drug exposure is from about 20 hr·ng/mL to about 150 hr·ng/mL.
